(19) United States
(12) Patent Application Publication
(10) Pub. No.: US 2018/0028262 A1
(43) Pub. Date: Feb. 1, 2018

(54) RF ABLATION NEEDLE

(71) Applicant: BOSTON SCIENTIFIC SCIMED, INC., Maple Grove, MN (US)

(72) Inventors: Hong CAO, Maple Grove, MN (US); Timothy Ostroot, Cokato, MN (US)

(21) Appl. No.: 15/653,189

(22) Filed: Jul. 18, 2017

Related U.S. Application Data
(60) Provisional application No. 62/367,468, filed on Jul. 27, 2016.

(51) Int. Cl. A61B 18/14

(52) U.S. Cl. CPC A61B 18/1477; A61B 18/1402; A61B 18/1492; A61B 2018/00005

(57) ABSTRACT

An ablation device includes a needle extending from a proximal end which, during use, remains outside a body accessible to a user to a tissue penetrating distal tip, the needle defining a lumen extending therethrough to a distal end of the needle. The ablation device also includes a first ablation electrode mounted on a distal portion of the needle in combination with an inner tube received within the lumen of the needle separating the lumen of the needle into an inner cooling fluid supply lumen and an annular fluid return lumen. In addition, the ablation device includes a source of cooling fluid supplying cooling fluid to the cooling fluid supply lumen and withdrawing fluid from the fluid return lumen to remove heat from the first electrode to maintain a temperature of the first electrode below a predetermined threshold temperature.

### PRIORITY CLAIM

The present disclosure claims priority to U.S. Provisional Patent Application Ser. No. 62/367,468 filed Jul. 27, 2016; the disclosure of which is incorporated herewith by reference.

### BACKGROUND

The present embodiments relate to systems and methods for ablating tissue in interior regions of the human body and, more particularly, to needle devices permitting tissue ablation and biopsy or target tissue. Radio-frequency (RF) probes have been used to treat tissue. However, these devices have been limited in certain applications by the size of the lesions they are able to create as well as the effects of the delivery of energy to non-targeted tissues.

### SUMMARY

The present disclosure relates to an ablation device which includes a needle extending from a proximal end which, during use, remains outside a body accessible to a user to a tissue penetrating distal tip, the needle defining a lumen extending therethrough to a distal end of the needle; an first ablation electrode mounted on a distal portion of the needle; an inner tube received within the lumen of the needle separating the lumen of the needle into an inner cooling fluid supply lumen and an annular fluid return lumen; and a source of cooling fluid supplying cooling fluid to the cooling fluid supply lumen and withdrawing fluid from the fluid return lumen to remove heat from the first electrode to maintain a temperature of the first electrode below a predetermined threshold temperature.

In an embodiment, the needle includes a distal opening and a plug sealing the distal opening to direct fluid from the fluid supply lumen into the fluid return lumen.

In an embodiment, the inner tube has a distal end separated proximally from the plug by a distance selected to provide fluid communication between the fluid supply lumen and the fluid return lumen adjacent to the first electrode.

In an embodiment, the inner tube is coupled to the plug and wherein the inner tube comprises at least one opening adjacent the first electrode providing fluid communication with the return lumen.

In an embodiment, the ablation device further includes an electrically insulative delivery element extending circumferentially around the needle and receiving the needle therein for movement between a retracted position in which the distal tip of the needle is received within the delivery element and an extended position in which the distal tip of the needle is projected distally beyond a distal end of the delivery element. The needle is formed of an electrically conductive material, the needle further comprising an electrically insulative coating extending circumferentially therearound, the coating extending along a portion of a length of the needle and having a distal end separated from the distal tip of the needle by a length selected to permit the portion of the needle extending distally from the coating to function as the first electrode, a proximal end of the coating being positioned so that, when the distal tip is projected distally beyond the distal end of the delivery element by a maximum distance, a part of the coating extends into the delivery element.

In an embodiment, the inner tube is slidably received within the needle so that, when the inner tube is withdrawn proximally from the needle, the plug is withdrawn from the distal tip of the needle opening the lumen of the needle to an exterior of the needle so that a tissue sample may be captured therein.

In an embodiment, the plug includes a sealing member extending circumferentially therearound to enhance a seal between the plug and the distal tip of the needle.

In an embodiment, the ablation device further includes a wall extending across and sealing a proximal part of the lumen of the needle from a distal portion thereof, the wall being spaced from a distal end of the lumen of the needle by a distance selected to form a tissue sample receiving space in the distal tip of the needle.

In an embodiment, the wall includes an opening therethrough and wherein the device includes a plunger member on a distal side of the wall and a control member extending from the plunger, through the opening to a proximal end accessible to a user so that, tension applied to the control member draws the plunger into contact with the wall to seal the opening and compression applied to the plunger forces the plunger distally away from the wall to drive tissue out of the tissue sample receiving space.

In an embodiment, the ablation device further includes a second electrode formed on a distal portion of the needle and separated longitudinally and electrically isolated from the first electrode, the first and second electrodes being coupled to opposite poles of a power source to function as a bi-polar ablation system.

In an embodiment, the needle is formed of an electrically conductive material and includes an electrically insulative coating circumferentially surrounding this electrically insulative coating along at least a portion thereof, and wherein the second electrode is mounted over the electrically insulative coating and the first electrode is mounted on the conductive material of the needle.

In an embodiment, a proximal portion of the needle is formed as a first electrically conductive tube and a distal end of the needle is formed as a second electrically conductive tube, the first and second electrically conductive tubes being joined to one another and electrically isolated from one another by an electrically insulative member.

In an embodiment, the ablation device further includes an electrically insulative delivery element extending circumferentially around the needle and receiving the needle therein for movement between a retracted position in which the distal tip of the needle is received within the delivery element and an extended position in which the distal tip of the needle is projected distally beyond a distal end of the delivery element; and an electrically insulative coating extending circumferentially around the first electrically conductive tube from a coating proximal end to a coating distal end, the coating proximal end being positioned so that, when the distal tip is projected distally beyond the distal end of the delivery element by a maximum distance, a part of the coating extends into the delivery element, a part of the first tube extending distally beyond a distal end of the coating forming the second electrode and the second tube forming the first electrode.

In an embodiment, the device is sufficiently flexible to be passed through a natural body lumen until a distal end of the needle reaches a target site within the body.

### BRIEF DESCRIPTION

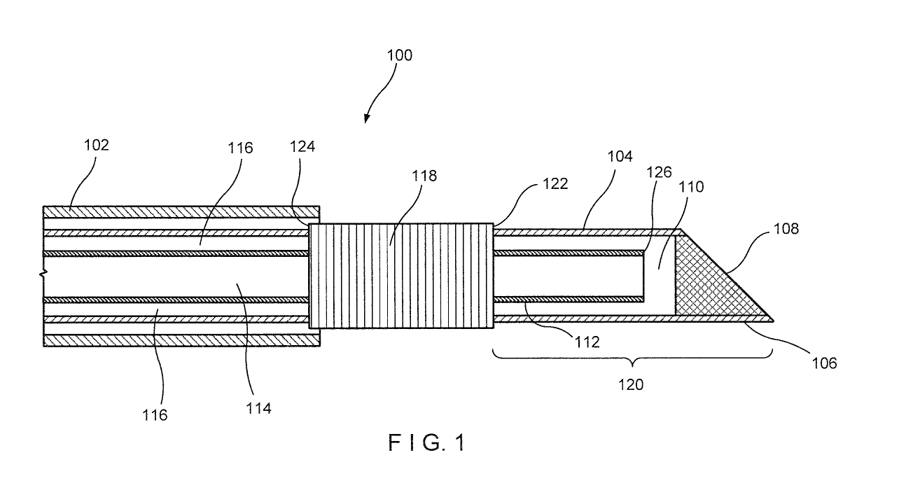

FIG. 1 shows a partially cross-sectional view of a monopolar RF ablation device according to an exemplary embodiment of the present disclosure;

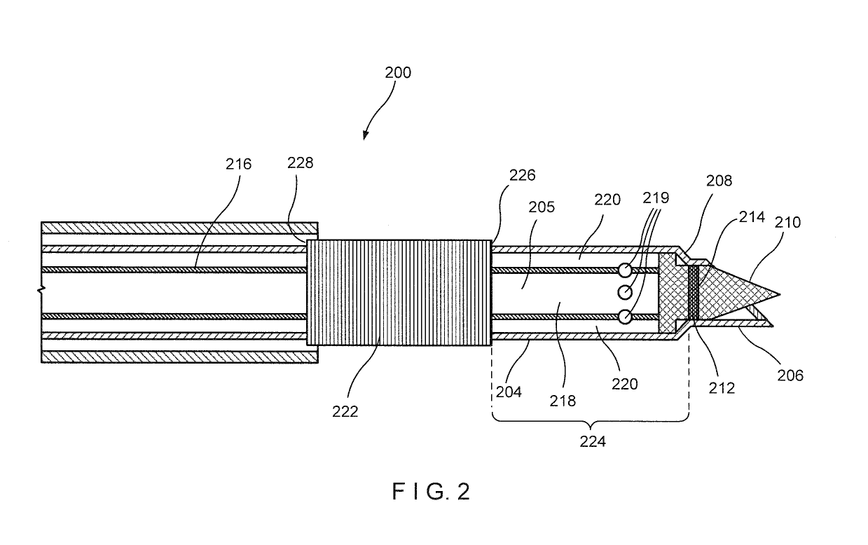

FIG. 2 shows a partially cross-sectional view of a monopolar RF ablation device according to a second exemplary embodiment of the present disclosure;

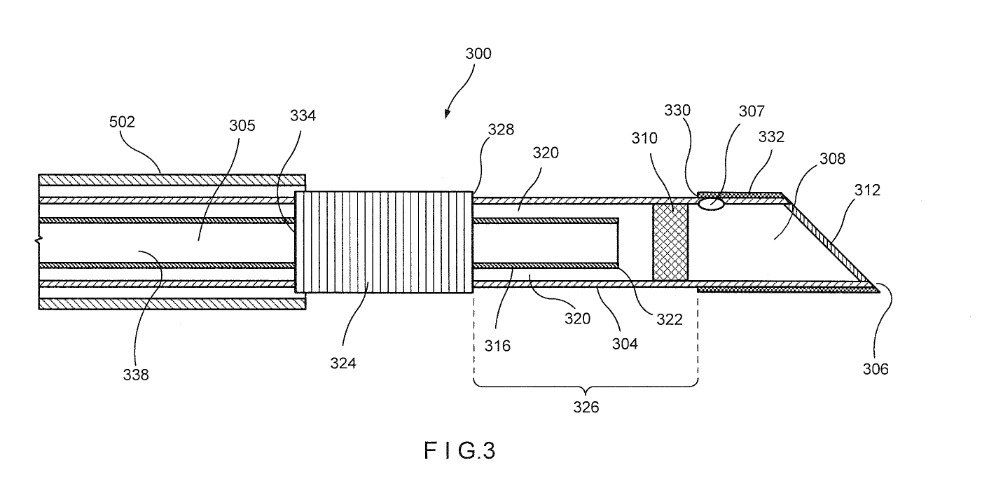

FIG. 3 shows a partially cross-sectional view of a monopolar RF ablation device according to a third exemplary embodiment of the present disclosure;

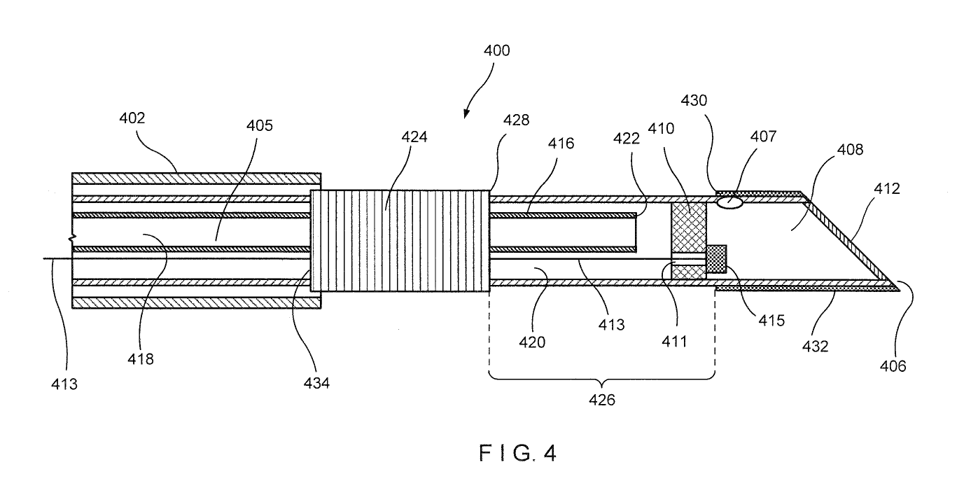

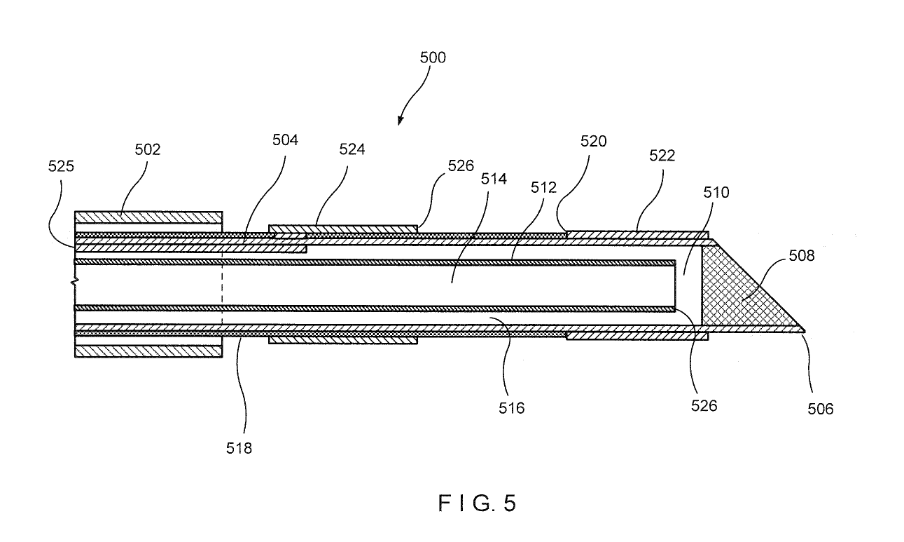

FIG. 4 shows a partially cross-sectional view of a monopolar RF ablation device according to a fourth exemplary embodiment of the present disclosure;

FIG. 5 shows a partially cross-sectional view of a bipolar RF ablation device according to a fifth exemplary embodiment of the present disclosure;

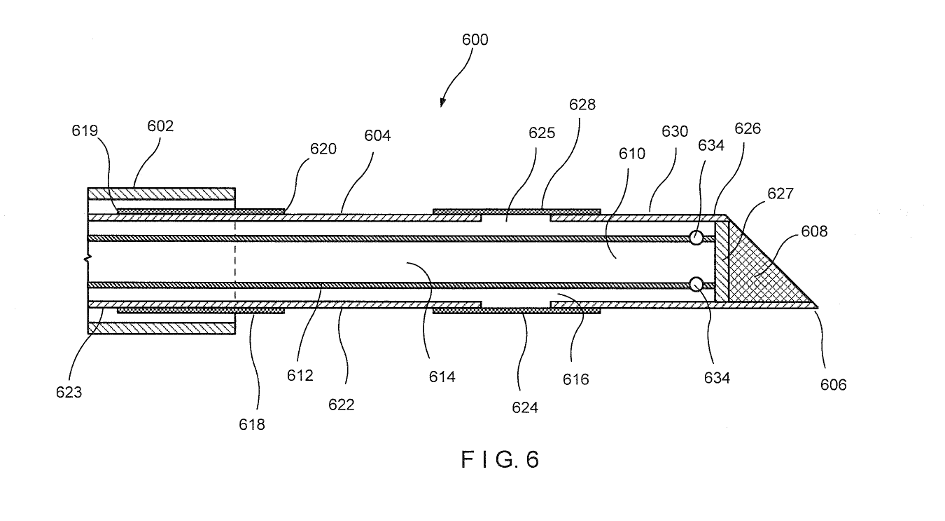

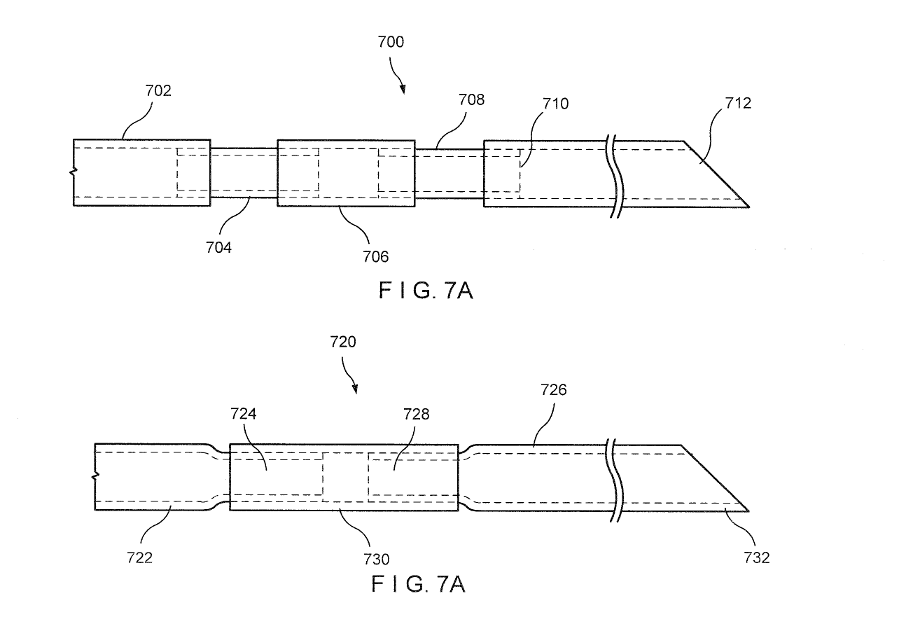

FIG. 6 shows a partially cross-sectional view of a bipolar RF ablation device according to a sixth exemplary embodiment of the present disclosure; and

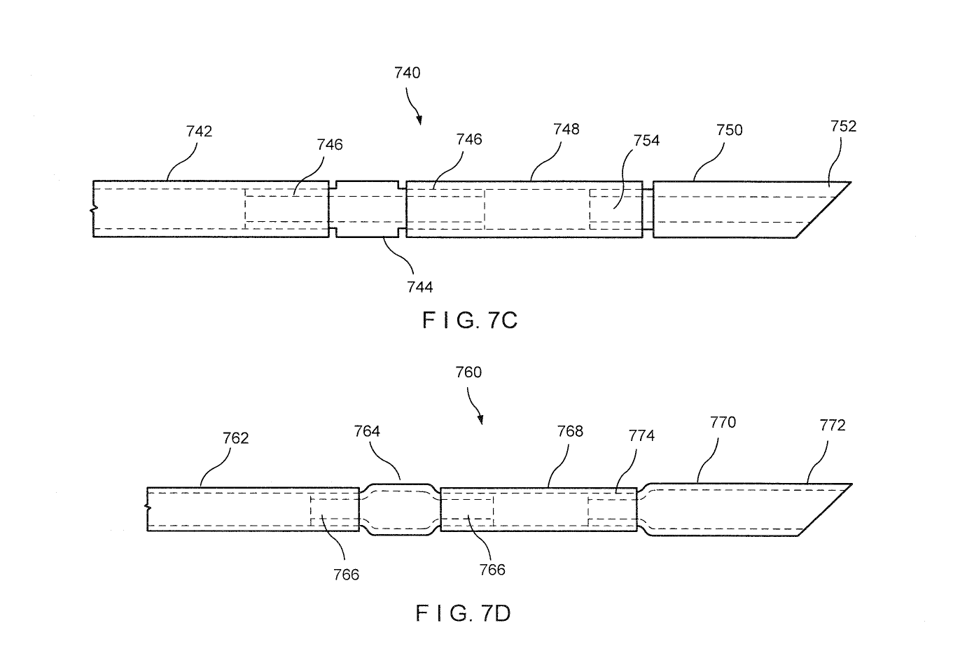

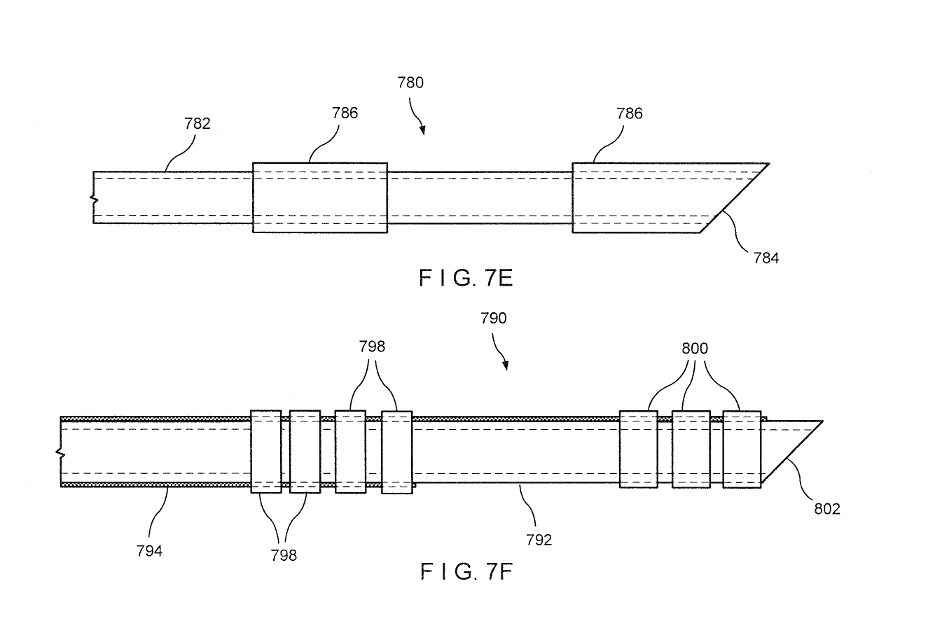

FIGS. 7A-7F show a partially cross-sectional views of a alternate constructions of bipolar RF ablation devices according to further embodiments of the present disclosure.

### DETAILED DESCRIPTION

The present disclosure may be further understood with reference to the appended drawings and the following description, wherein like elements are referred to with the same reference numerals. The present disclosure relates to devices and methods for ablating tissue and, more particularly, relates to needle devices for ablating tissue and/or collecting tissue samples. It should be noted that the terms proximal and distal, as used herein, are intended to refer to a direction toward (proximal) and away from (distal) a user of the device (e.g., physician).

As shown in FIG. 1, a device 100 comprises an electrically insulative delivery element 102 within which an electrically conductive ablation needle 104 is slidably received. The delivery element 102 according to this embodiment may be sized, for example, to pass through the working channel of an endoscope or bronchoscope for delivery to target tissue within a living body. As would be understood by those skilled in the art, the device 100 is preferably sufficiently flexible to pass through a tortuous path through, for example, a natural body lumen without undue trauma to tissue along and adjacent to the lumen or damage to the device 100. For example, the device 100 may have a flexibility sufficient to permit the device 100 to be slidably inserted through a working channel of a device such as a flexible endoscope or bronchoscope and to pass through any bending radii that these devices may achieve. The delivery element 102 may be formed as a flexible sheath similar to those currently employed for flexible biopsy needles and defines an internal lumen 106 within which the ablation needle 104 is slidably received. In an exemplary embodiment, the delivery element is formed as a sheath of polyether ether ketone (PEEK) having an outer diameter of 0.68″. However, as would be understood by those skilled in the art, other materials and sizes may be used. The ablation needle 104 of this embodiment is formed of a flexible, biocompatible and electrically conductive material such as stainless steel, nitinol, Inconel, platinum and other biocompatible electrically conductive materials. According to the exemplary embodiment, the needle 104 may be formed, for example, as a stainless steel hypotube having an outer diameter of 0.045″ with an inner diameter of 0.037″. Those skilled in the art will understand that these dimensions are exemplary only and other dimensions may be used as desired.

The needle 104 of the exemplary embodiment extends between a proximal end which, in use, remains outside the body accessible to a user, and a tissue penetrating distal tip 106. The needle 104 includes a distal plug 108 that seals the distal end of a lumen 110 of the needle 104. Those skilled in the art will understand that, in this embodiment, the distal tip 106 of the needle is ground to form a tissue penetrating tip and that the distal end of the ground tube opens the lumen 110 of the needle 104 to the external environment. In this embodiment, this distal opening is sealed by a separate plug element 108 that may be formed of any suitable material such as, for example, metal, rubber or plastic. Alternatively, the end of the needle 104 may be sealed by the same material of which the needle is formed (e.g., stainless steel). An inner tube 112 divides the lumen 110 into a central passage 114 and an outer, annular channel 116. The inner tube 112 may be formed, for example, as a stainless steel hypotube, a polyimide tube or a nylon tube having, for example, an outer diameter of 0.025″ and an inner diameter of 0.020″. An electrically insulating layer 118 extends circumferentially around a portion of the needle 104. The layer 118 is separated from the distal tip 106 by a distance selected as a length of a portion of the needle 104 that serves as an ablation electrode 120. The electrically insulative layer 118 may, for example, be formed as a heat shrunk layer of polyethylene terephthalate (PET) with a thickness of 0.0005″. Those skilled in the art will understand that the materials and dimensions provided are exemplary and other materials and sizes may be used depending on the requirements of specific procedures to be performed.

Specifically, the portion 120 of the needle 104 extending distally from the distal end 122 of the electrically insulating layer 118 to the distal tip 206 receives electrical energy supplied to a proximal end of the needle 104 for ablating target tissue adjacent to the portion 120. The electrically insulative layer 118 preferably has a length selected so that, when the needle 104 is extended distally from the delivery element 102 by a maximum extent, a proximal end 124 of the electrically insulative layer 118 remains within the delivery element 102 to ensure that energy is delivered to tissue only via the portion 120. Those skilled in the art will understand that any known mechanisms may be employed to advance and retract the needle 104 relative to the delivery element 102 and that any known power source and controller may be used to supply the RF ablation energy to the portion 120. For example, a Boston Scientific RF3000™ may be used as the power source for any of the embodiments described herein while a Boston Scientific MetriQ™ pump may be used to supply the cooling fluid in any of the disclosed embodiments.

During use, cooling fluid (e.g., sterile saline or water) is supplied to the portion 120 of the needle 104 to maintain a temperature of the portion 120 below a desired threshold level. In an exemplary embodiment, this may be the temperature at which tissue charring occurs. For example, a flow rate of, for example, 10-40 ml/minute, either at room temperature or chilled below room temperature, may be maintained to control the temperature as desired to maintain the temperature of the portion 120 below 100 degrees C. to avoid tissue charring. Those skilled in the art would understand that feedback may be obtained from a thermocouple, thermistor or other sensor allowing the system or the user to adjust a flow rate of cooling fluid to maintain a desired temperature of the portion 120. Those skilled in the art will understand that the temperature of the portion 120 may also be monitored based on a differential between a temperature of fluid supplied to the system and a temperature of the fluid withdrawn from the system after cooling the portion 120. In one embodiment, the system 100 (e.g., the power supply) controls power supply based on feedback relating to impendance measured between the portion 120 and a grounding patch on the patient (as would be understood by those skilled in the art) which is related to the temperature at the portion 120. In some cases, preventing the charring of surrounding tissue permits the ablation needle 104 according to this embodiment to ablate larger volumes of tissue as desiccated charred tissue does not efficiently conduct electrical energy. That is, because of the higher electroconductivity of the non-charred tissue, energy can be delivered to larger amounts of tissue by preventing charring. Thus, the needle 104 may create lesions up to 4 cm or larger in diameter which is suitable for the tumor treatment. In use, the tissue penetrating distal tip 106 may be used to penetrate a target portion of tissue (e.g., a tumor). Using any known visualization system, a user may determine when the needle 104 is in a desired position with, for example, the portion 120 centered within a target portion of tissue to be ablated. At this point, power may be supplied to the portion 120 while cooling fluid is supplied to the inner tube 112 as necessary to maintain the desired temperature of the portion 120. The cooling fluid flows distally out of a distal end 126 of the tube 112 and, as the distal end of the needle 104 is sealed by the distal plug 108, the fluid enters the annular channel 116 through which it is withdrawn proximally from the portion 120 of the needle 104 carrying away heat. The fluid may be withdrawn from the needle 104 via the annular channel 116 or cooled and recirculated as would be understood by those skilled in the art. In another exemplary embodiment, the direction of flow of the cooling fluid may be reversed. For example, the cooling fluid may be supplied to the annular channel 116 to maintain a desired temperature of the portion 120 and withdrawn proximally through the inner tube 112. When the target portion of tissue has been ablated as desired, the power supply and flow of cooling fluid are terminated and the needle 104 is withdrawn proximally until the tissue penetrating distal tip 106 is received within the delivery element 102. The device 100 may then be withdrawn from the body or moved to a new position adjacent to or within a second target portion of tissue to be ablated. The process may then be repeated as desired.

As shown in FIG. 2, a needle ablation device 200 according to a further embodiment can be used to ablate tissue in a manner similar to that described above in regard to the device 100 and can also be used for aspiration biopsy as will be described below. The device 200 includes an electrically insulative, flexible delivery element 202 which may be substantially the same as the delivery element 102 of the device 100 with an electrically conductive ablation needle 204 slidably received therein. The ablation needle 204 of this embodiment may be formed of the same materials and in the same or different dimensions as described above for the needle 104 as may be dictated by the procedure for which the needle is to be used.

The needle 204 of this embodiment extends between a proximal end which, in use, remains outside the body accessible to a user, and a tissue penetrating distal tip 206. The needle 204 defines a lumen 205 therein and includes a tapered connection 208 between a proximal portion of the needle 204 and the distal tip 206. A plug 210 is movable between a first position in which it seals a distal opening 212 of the needle 204 and a second position in which the plug 210 is withdrawn from the opening 212 so that tissue samples may be captured within the needle 204 for biopsies as will be described below. The plug 210 in this embodiment includes an optional O-ring 214 to enhance the seal created by the plug 210 when it is in the first position within the opening 212.

An inner tube 216 divides the lumen 205 into a central passage 218 and an outer, annular channel 220. The inner tube 216 may be formed of the same materials and dimensions described above in regard to the inner tube 112 and extends from a proximal end that, in use remains accessible to a user to a distal end coupled to the plug 210. The inner tube 216 includes one or more openings 219 adjacent to a distal end thereof to permit cooling fluid supplied to the central passage 218 to pass through the tube 216 into the annular channel 220 to cool the distal portion of the needle 204 as desired. The inner tube 216 is slidably received within the needle 204 so that, during insertion to a target site within the body, the plug 210 seals the opening 212 until a user desires to obtain a tissue sample. At this point (e.g., when the needle 204 is adjacent to a portion of tissue to be sampled), the user can withdraw the inner tube 216 proximally to remove the plug 210 from the opening 212 and expose the lumen 205. The inner tube 216 and the plug 210 may be withdrawn by any distance desired to permit the capture of a desired tissue sample. As would be understood by those skilled in the art, if desired the inner tube 216 and the plug 210 may be fully withdrawn from the needle 204 permitting tissue captured in the distal portion of the needle 204 to be aspirated out of the proximal end of the needle 204 for study. An electrically insulating layer 222 extends circumferentially around a portion of the needle 204 separated from the distal tip 206 by a distance selected as a length of a portion of the needle 204 that serves as an ablation electrode 224. The electrically insulative layer 222 may, for example, be similar in material and dimension to the layer 118 described above. Those skilled in the art will understand that the materials and dimensions provided are exemplary and other materials and sizes may be used depending on the requirements of specific procedures to be performed.

Specifically, the portion 224 of the needle 204 extending distally from the distal end 226 of the electrically insulating layer 222 to the tapered connection 208 receives electrical energy supplied to a proximal end of the needle 204 for ablating target tissue adjacent to the portion 224. The electrically insulative layer 222 preferably has a length selected so that, when the needle 204 is extended distally from the delivery element 202 by a maximum extent, a proximal end 228 of the electrically insulative layer 222 remains within the delivery element 202 to ensure that energy is delivered to tissue only via the portion 224. Those skilled in the art will understand that any known mechanisms may be employed to advance and retract the needle 204 relative to the delivery element 202 and that any known power source and controller may be used to supply the RF ablation energy to the portion 224.

During use, cooling fluid (e.g., sterile saline) is supplied to the portion 220 of the needle 204 to maintain a temperature of the portion 224 below a threshold level. Those skilled in the art will understand that the desired temperature ranges and the desired amounts of power described above in regards to the device 100 will be substantially similar for the device 200. Thus, the needle 204 may also create lesions up to 4 cm or larger in diameter which is suitable for the tumor treatment. In use, the inner tube 216 is maintained in the distal position with the plug 210 sealing the opening 212 until a desired position has been reached adjacent to or within tissue to be ablated. Specifically, the tissue penetrating distal tip 206 may be used to penetrate a target portion of tissue (e.g., a tumor) while the user (e.g., using a known visualization system) determines when the needle 204 is in a desired position with, for example, the portion 224 centered within a target portion of tissue to be ablated. At this point, power may be supplied to the portion 224 while cooling fluid is supplied to the inner tube 216 as necessary to maintain the desired temperature of the portion 224. The cooling fluid flows out of the openings 219 into the annular channel 220 and, as the 212 is sealed by the distal plug 210, the fluid enters the annular channel 220 through which it is withdrawn proximally from the portion 224 of the needle 204 carrying away heat. The fluid may be withdrawn from the needle 204 via the annular channel 220 or cooled and recirculated via the central passage 218 as would be understood by those skilled in the art. When the target portion of tissue has been ablated as desired, the power supply and flow of cooling fluid are terminated and the needle 204 is withdrawn proximally until the tissue penetrating distal tip 206 is received within the delivery element 202. The device 200 may then be withdrawn from the body or moved to a new position adjacent to or within a second target portion of tissue to be ablated. The process may then be repeated as desired.

When a user wishes to use the device 200 to obtain a tissue sample, the user moves the device 200 to a location adjacent to the tissue to be sampled (e.g., with the inner tube 216 and the plug 210 in the distal position sealing the opening 212. The user then withdraws the inner tube 216 and the plug 210 proximally through the lumen 205 until the plug 210 has been withdrawn a desired distance into the lumen 205 or removed entirely therefrom as desired. The user then advances the distal tip 206 into the target tissue to capture a tissue sample therein as would be understood by those skilled in the art. This tissue sample may then be aspirated from the needle 204 by providing suction to the lumen 205. After the sample has been aspirated from the lumen 205, additional samples may be captured in the same manner. As would be understood by those skilled in the art, the inner tube 216 may then be reinserted as desired to perform additional ablations or to reposition the needle 204 for sampling of tissue at a different location.

As seen in FIG. 3, a device 300 is substantially similar to the device 200 described above except as described below. The device 300 includes an electrically insulative, flexible delivery element 302 which may be substantially the same as the delivery elements 102 and 202 with an electrically conductive ablation needle 304 slidably received therein. The ablation needle 304 of this embodiment may be formed of the same materials and in the same or different dimensions as described above for the needles 104, 204 as may be dictated by the procedure for which the needle is to be used.

The needle 304 of this embodiment extends between a proximal end which, in use, remains outside the body accessible to a user, and a tissue penetrating distal tip 306. The needle 304 defines a lumen 305 therein and extends to a distal tip 306. A wall 310 is mounted in the lumen 305 at a position proximal of the distal tip 306 by a distance selected to create a tissue receiving space 308 in a portion of the lumen 305 between a distal opening 312 of the needle 304 and distal of the of the wall 310. Thus, the portion of the lumen 305 proximal to the wall 310 is sealed with respect to the space 308 and the environment external to the needle 304. In this embodiment, the needle 304 further includes a vent hole 307 located just distally of the wall 310 and open to the receiving space 308 to allow fluid to flow out of the receiving space 308 when target tissue is pierced.

An inner tube 316 divides the lumen 305 into a central passage 318 and an outer, annular channel 320. The inner tube 316 may be formed of the same materials and dimensions described above in regards to the inner tubes 112, 216 and extends from a proximal end that, in use remains accessible to a user to a distal end 322 separated from a proximal side of the wall 310 by a space open to the annular channel 320. The inner tube 316 may be permanently mounted within the needle 304 to maintain the space between the distal end 322 and the wall 310. A first electrically insulating layer 324 extends circumferentially around a portion of the needle 304 with a portion of the needle 304 extending distally therefrom to serve as an ablation electrode 326. Specifically, in this embodiment, the electrode 326 extends from a distal end 328 of the first layer 324 to a proximal end 330 of a second electrically insulative layer 332. In this embodiment, the second layer 332 covers a portion of the needle 304 extending distally of the wall 310 so that substantially an entire length of the electrode 326 can be cooled by the cooling fluid circulating in the lumen 305. However, those skilled in the art will understand that the second layer 332 may be extended proximally or distally or eliminated entirely as necessary to achieve the desired temperature range of the electrode 326 which will be substantially similar to that described in regard to the device 100. The electrically insulative layers 324, 332 may, for example, be similar in material and dimension to the layers 118, 222 described above. Those skilled in the art will understand that the materials and dimensions provided are exemplary and other materials and sizes may be used depending on the requirements of specific procedures to be performed.

Specifically, the electrode 326 receives electrical energy supplied to a proximal end of the needle 304 for ablating target tissue adjacent to the electrode 326. The first electrically insulative layer 324 preferably has a length selected so that, when the needle 304 is extended distally from the delivery element 302 by a maximum extent, a proximal end 334 of the first electrically insulative layer 324 remains within the delivery element 302 to ensure that energy is delivered to tissue only via the electrode 326. That is, similar to the devices 100 and 200, electrically conductive portions of the needle 304 extending proximally from the proximal end 334 of the first electrically insulative layer 324 remain within the delivery element 302 to prevent current from being delivered from this proximal portion of the needle 302 to non-targeted tissue. Those skilled in the art will understand that any known mechanisms may be employed to advance and retract the needle 304 relative to the delivery element 302 and that any known power source and controller may be used to supply the RF ablation energy to the portion 326.

During use, cooling fluid (e.g., sterile saline) is supplied to the electrode 326 to maintain a temperature of the electrode 326 below a threshold level. Those skilled in the art will understand that the desired temperature ranges and the desired amounts of power described above in regards to the device 100, 200 will be substantially similar for the device 300. Thus, the needle 304 may also create lesions up to 4 cm or larger in diameter which is suitable for the tumor treatment. In use, the needle 304 is withdrawn proximally into the delivery element 302 and the device is advanced (e.g., through an endoscope or bronchoscope) to a position in the body adjacent to tissue to be ablated. The needle 304 is then advanced distally from the distal end of the delivery element 302 to expose the distal tip 306. The tissue penetrating distal tip 306 may then be inserted into a target portion of tissue (e.g., a tumor) to capture a tissue sample and/or to position the electrode 326 as desired within a mass of tissue to be ablated. When the tissue penetrating distal tip 306 is inserted into the target portion of tissue, any fluid retained within the tissue receiving space 308 may be forced out of the receiving space 308 through vent hole 307 by the target tissue. This flow of the fluid out of the distal end of the needle 304 prevents the tissue receiving space 308 from hydrolocking and allows tissue to be received within the tissue receiving space 308. When the user has determined that the electrode 326 is at a desired position within or adjacent to tissue to be ablated (e.g., using a known visualization system), power may be supplied to the electrode 326 while cooling fluid is supplied to the inner tube 316 as necessary to maintain the desired temperature of the electrode 326. The cooling fluid flows out of the distal end of the central passage 318 and into the annular channel 320 through which it is withdrawn proximally from the electrode 326 carrying away heat to maintain the temperature of the electrode 326 in a desired range. The fluid may be withdrawn from the needle 304 via the annular channel 320 or cooled and recirculated via the central passage 318 as would be understood by those skilled in the art. When the target portion of tissue has been ablated as desired, the power supply and flow of cooling fluid are terminated and the needle 304 is withdrawn proximally until the tissue penetrating distal tip 306 is received within the delivery element 302. The device 300 may then be withdrawn from the body or moved to a new position adjacent to or within a second target portion of tissue to be ablated. The process may then be repeated as desired. Any tissue sample collected in the tissue receiving space 308 may be retrieved when the device 300 is withdrawn from the body.

As seen in FIG. 4, a device 400 is substantially similar to the device 300 described above except for the inclusion of a plunger as described below. The device 400 includes an electrically insulative, flexible delivery element 402 which may be substantially the same as the delivery elements 102, 202, 302 with an electrically conductive ablation needle 404 slidably received therein. The ablation needle 404 of this embodiment may be formed of the same materials and in the same or different dimensions as described above for the needles 104, 204, 304 as may be dictated by the procedure for which the needle is to be used.

The needle 404 of this embodiment extends between a proximal end which, in use, remains outside the body accessible to a user, and a tissue penetrating distal tip 406. The needle 404 defines a lumen 405 therein and extends to a distal tip 406. A wall 410 is mounted in the lumen 405 at a position proximal of the distal tip 406 by a distance selected to create a tissue receiving space 408 in a portion of the lumen 405 between a distal opening 412 of the needle 404 and distal of the of the wall 410. Thus, the portion of the lumen 405 proximal to the wall 410 is sealed with respect to the space 408 and the environment external to the needle 404. In this embodiment, the needle 404 further includes a vent hole 407 located just distally of the wall 410 and open to the receiving space 408 to allow fluid to flow out of the receiving space 408 when target tissue is pierced.

An inner tube 416 divides the lumen 405 into a central passage 418 and an outer, annular channel 420. The inner tube 416 may be formed of the same materials and dimensions described above in regards to the inner tubes 112, 216, 316 and extends from a proximal end that, in use remains outside the body accessible to a user to a distal end 422 separated from a proximal side of the wall 410 by a space open to the annular channel 420. The inner tube 416 may be permanently mounted within the needle 404 to maintain the space between the distal end 422 and the wall 410. A first electrically insulating layer 424 extends circumferentially around a portion of the needle 404 with a portion of the needle 404 extending distally therefrom to serve as an ablation electrode 426. Specifically, in this embodiment, the electrode 426 extends from a distal end 428 of the first layer 424 to a proximal end 430 of a second electrically insulative layer 432. In this embodiment, the second layer 432 covers a portion of the needle 404 extending distally of the wall 410 so that substantially an entire length of the electrode 426 can be cooled by the cooling fluid circulating in the lumen 405. However, those skilled in the art will understand that the second layer 432 may be extended proximally or distally or eliminated entirely as necessary to achieve the desired temperature range of the electrode 426 which will be substantially similar to that described in regard to the devices 100, 200 and 300. The electrically insulative layers 424, 432 may, for example, be similar in material and dimension to the layers 118, 222, 324 described above. Those skilled in the art will understand that the materials and dimensions provided are exemplary and other materials and sizes may be used depending on the requirements of specific procedures to be performed.

Specifically, the electrode 426 receives electrical energy supplied to a proximal end of the needle 404 for ablating target tissue adjacent to the electrode 426. The first electrically insulative layer 424 preferably has a length selected so that, when the needle 404 is extended distally from the delivery element 402 by a maximum extent, a proximal end 434 of the first electrically insulative layer 424 remains within the delivery element 402 to ensure that energy is delivered to tissue only via the electrode 426. That is, similar to the devices 100, 200 and 300, electrically conductive portions of the needle 404 extending proximally from the proximal end 434 of the first electrically insulative layer 424 remain within the delivery element 402 to prevent current from being delivered from this proximal portion of the needle 402 to non-targeted tissue. Those skilled in the art will understand that any known mechanisms may be employed to advance and retract the needle 404 relative to the delivery element 402 and that any known power source and controller may be used to supply the RF ablation energy to the portion 426.

In contrast to the device 300, the wall 410 of the device 400 includes an opening 411 extending therethrough. A control member 413 extends within the lumen 405 from a proximal end that remains accessible to a user (e.g., via an actuator coupled to a device handle (Not shown)) and passes through the opening 411. A distal end of the control member 413 is coupled to a plunger 415. The plunger 415 can be operated by a user to seal the opening 411 to facilitate the flow of cooling fluid from the central passage 418 to the annular channel 420 by applying tension to the control member 413. In addition, the user can operate the plunger 415 to force a captured tissue sample out of the space 408 by advancing the control member 413 distally into the lumen 405 to push the plunger 415 distally into the space 408 thereby forcing tissue stored in the space 408 out of the distal opening 412 for retrieval and analysis.

During use, cooling fluid (e.g., sterile saline) is supplied to the electrode 426 to maintain a temperature of the electrode 426 below a threshold level. Those skilled in the art will understand that the desired temperature ranges and the desired amounts of power described above in regards to the device 100, 200, 300 will be substantially similar for the device 400. Thus, the needle 404 may also create lesions up to 4 cm or larger in diameter which is suitable for the tumor treatment. In use, the needle 404 is withdrawn proximally into the delivery element 402 and the device is advanced (e.g., through an endoscope or bronchoscope) to a position in the body adjacent to tissue to be ablated. The needle 404 is then advanced distally from the distal end of the delivery element 302 to expose the distal tip 406. The tissue penetrating distal tip 406 may then be inserted into a target portion of tissue (e.g., a tumor) to capture a tissue sample and/or to position the electrode 426 as desired within a mass of tissue to be ablated. When the distal tip 406 is inserted into the target portion of tissue, any fluid retained within the tissue receiving space 408 may be forced out of the receiving space 408 through vent hole 407 by the target tissue. This flow of the fluid out of the distal end of the needle 404 prevents the tissue receiving space 408 from hydrolocking and allows tissue to be received within the tissue receiving space 408. When the user has determined that the electrode 426 is at a desired position within or adjacent to tissue to be ablated (e.g., using a known visualization system), the control member 413 may be drawn proximally to seal the opening 411 and power may be supplied to the electrode 426 while cooling fluid is supplied to the inner tube 416 as necessary to maintain the desired temperature of the electrode 426. The cooling fluid flows out of the distal end of the central passage 418 and into the annular channel 420 through which it is withdrawn proximally from the electrode 426 carrying away heat to maintain the temperature of the electrode 426 in a desired range. The fluid may be withdrawn from the needle 404 via the annular channel 420 or cooled and recirculated via the central passage 418 as would be understood by those skilled in the art. When the target portion of tissue has been ablated as desired, the power supply and flow of cooling fluid are terminated and the needle 404 is withdrawn proximally until the tissue penetrating distal tip 406 is received within the delivery element 402. The device 400 may then be withdrawn from the body or moved to a new position adjacent to or within a second target portion of tissue to be ablated. The process may then be repeated as desired. Any tissue sample collected in the tissue receiving space 408 may be retrieved when the device 400 is withdrawn from the body by advancing the control member 413 distally to force the plunger 415 distally through the space 408, thereby ejecting the tissue collected therein.

As seen in FIG. 5, a device 500 is substantially similar to the device 100 and comprises an electrically insulative, flexible delivery element 502 within which an electrically conductive ablation needle 504 is slidably received. The delivery element 502 according to this embodiment may be sized, for example, to pass through the working channel of an endoscope or bronchoscope for delivery to target tissue within a living body. As would be understood by those skilled in the art, the device 500 is preferably sufficiently flexible to pass through a tortuous path through, for example, a natural body lumen without undue trauma to tissue along and adjacent to the lumen or damage to the device 500. For example, the device 500 may have a flexibility sufficient to permit the device 500 to be slidably inserted through a working channel of a device such as a flexible endoscope or bronchoscope and to pass through any bending radii that these devices may achieve. The delivery element 502 may be formed as a flexible sheath similar to those currently employed for flexible biopsy needles and defines an internal lumen 506 for slidably receiving ablation needle 504. In an exemplary embodiment, the delivery element 502 is formed as a sheath of polyether ether ketone (PEEK) having an outer diameter of 0.68″. However, as would be understood by those skilled in the art, other materials and sizes may be used. The ablation needle 504 of this embodiment is formed of a flexible, biocompatible and electrically conductive material such as stainless steel, nitinol, Inconel, platinum and other biocompatible electrically conductive materials. According to the exemplary embodiment, the needle 504 may be formed, for example, as a stainless steel hypotube having an outer diameter of 0.045″ with an inner diameter of 0.037″.

The needle 504 of the exemplary embodiment extends between a proximal end which, in use, remains outside the body accessible to a user, and a tissue penetrating distal tip 506. The needle 504 includes a distal plug 508 that seals the distal end of a lumen 510 of the needle 504. Those skilled in the art will understand that, in this embodiment, the distal tip 506 of the needle is ground to form a tissue penetrating tip and that the distal end of the ground tube opens the lumen 510 of the needle 504 to the external environment. In this embodiment, this distal opening is sealed by a separate plug element 508 that may be formed of any suitable material such as, for example, metal, rubber or plastic. Alternatively, the end of the needle 504 may be sealed by the same material of which the needle 504 is formed (e.g., stainless steel). An inner tube 512 divides the lumen 510 into a central passage 514 and an outer, annular channel 516. The inner tube 512 may be formed, for example, as a stainless steel hypotube, a polyimide tube or a nylon tube having, for example, an outer diameter of 0.025″ and an inner diameter of 0.020″. An electrically insulating layer 518 extends distally around a portion of the needle 504 extending from a proximal end on a portion of the needle 504 that always remains within the delivery element 502 even when the needle 504 is extended distally therefrom by a maximum amount. Thus, the layer 518 prevents the delivery of electric energy to non-targeted portions of tissue that may come into contact with the needle 504. The layer 518 wraps circumferentially around the needle 504 and extends distally to a proximal end 520 of a first electrode 522. The first electrode 522 that extends circumferentially around a distal portion of the needle 504 adjacent to the distal tip 506. The first electrode is in contact with the electrically conductive material of the needle 504 to receive electrical energy directly therefrom. A second electrode 524 extends circumferentially around a portion of the needle 504 proximal of the first electrode 522. A distal end 526 of the second electrode 524 is separated from the proximal end 520 of the first electrode 522 by a distance selected to electrically isolate the first electrode 522 from the second electrode 524. The first and second electrodes 522, 524, respectively, are separately coupled to a source of electrical potential of opposite polarity to form a bipolar ablation device as would be understood by those skilled in the art. The second electrode 524 is coupled to an electrode 525 which runs inside or outside the needle 504 and is electrically isolated therefrom. The electrode 525 extends from a proximal end (not shown) coupled to a power source and passes through the needle 504 to couple to the second electrode 524. Those skilled in the art will understand that the electrode 525 may, alternatively, extend to the second electrode 524 outside the needle 504 within the delivery element 502.

As would be understood by those skilled in the art, a size of the first and second electrodes 522, 524, respectively, will be selected based on the application parameters such as the size of the lesion that is desired, an amount of power that can safely be delivered and constraints of the anatomy. In this embodiment, the first and second electrodes 522, 524, respectively, are each 1 cm in length and are separated from one another by a gap of 1 cm. Each of the first and second electrodes 522, 524, respectively, extends around the circumference of the needle 504 which, in this embodiment, has an outer diameter of approximately 0.045″. In addition, the layer 518 in this embodiment is formed as a 0.005″ thick heat shrunk layer of PET although other suitable materials may be employed. Those skilled in the art will understand that the materials and dimensions provided are exemplary and other materials and sizes may be used depending on the requirements of specific procedures to be performed. In addition, those skilled in the art will understand that any known mechanisms may be employed to advance and retract the needle 504 relative to the delivery element 502 and that any known power source and controller may be used to supply the RF ablation energy to the portion 520. As the second electrode 524 rests on the electrically insulative layer 518, the second electrode 524 may be made longer to allow power dispersal from the second electrode 524 to equal that from the first electrode 522. As would be understood by those skilled in the art, the addition of the layer 518 between a longer second electrode 524 and the needle 504 results in a lower current density with compensated for by an increased surface area.

During use, cooling fluid (e.g., sterile saline) is supplied to the distal portion of the needle 504 (i.e., the areas adjacent to both the first and second electrodes 522, 524) to maintain a temperature of this portion of the needle 504 below a desired threshold level. In an exemplary embodiment, this may be the temperature at which tissue charring occurs. As described above, by preventing the charring of surrounding tissue, the ablation needle 504 according to this embodiment can ablate larger volumes of tissue as desiccated charred tissue does not efficiently conduct electrical energy. Thus, the needle 104 may create lesions up to 4 cm or larger in diameter which is suitable for the tumor treatment. In use, the tissue penetrating distal tip 506 may be used to penetrate a target portion of tissue (e.g., a tumor). Using any known visualization system, a user may determine when the needle 504 is in a desired position with, for example, the first and second electrodes 522, 524 centered within a target portion of tissue to be ablated. At this point, power may be supplied to the first and second electrodes 522, 524 while cooling fluid is supplied to the inner tube 512 as necessary to maintain the desired temperature of the distal portion of the needle 504. The cooling fluid flows distally out of a distal end 526 of the tube 512 and, as the distal end of the needle 504 is sealed by the distal plug 508, the fluid enters the annular channel 516 through which it is withdrawn proximally from the portion 520 of the needle 504 carrying away heat. The fluid may be withdrawn from the needle 504 via the annular channel 516 or cooled and recirculated as would be understood by those skilled in the art. When the target portion of tissue has been ablated as desired, the power supply and flow of cooling fluid are terminated and the needle 504 is withdrawn proximally until the tissue penetrating distal tip 506 is received within the delivery element 502. The device 500 may then be withdrawn from the body or moved to a new position adjacent to or within a second target portion of tissue to be ablated. The process may then be repeated as desired.

As shown in FIG. 6, a bipolar ablation device 600 according to a further embodiment is substantially similar to the device 500 except as described below. The device 600 is substantially similar to the device 500 and comprises an electrically insulative, flexible delivery element 602 within which an electrically conductive ablation needle 604 is slidably received. The delivery element 602 according to this embodiment may be sized, for example, to pass through the working channel of an endoscope or bronchoscope for delivery to target tissue within a living body. Furthermore, those skilled in the art will understand that a non-conductive cooling fluid such as, for example, distilled water may be desirable in this embodiment.

The needle 604 of the exemplary embodiment extends between a proximal end which, in use, remains outside the body accessible to a user, and a tissue penetrating distal tip 606. The needle 604 includes a distal plug 608 that seals the distal end of a lumen 610 of the needle 604. Those skilled in the art will understand that, in this embodiment, the distal tip 606 of the needle 604 is ground to form a tissue penetrating tip and that the distal end of the ground tube opens the lumen 610 of the needle 604 to the external environment. In this embodiment, this distal opening is sealed by a separate plug element 608. An inner tube 612 divides the lumen 610 into a central passage 614 and an outer, annular channel 616. A first electrically insulating layer 618 extends wraps circumferentially around and extends distally along a portion of the needle 604 extending from a proximal end 619 on a portion of the needle 604 that always remains within the delivery element 602 even when the needle 604 is extended distally therefrom by a maximum amount. The layer 618 extends to a distal end 620. Distally of the distal end 620 of the first layer 618 the electrically conductive material of the needle 604 is exposed to form a first electrode 622. The electrically conductive portion 623 of the needle 604 ends distally just beyond the first electrode 622. At this point, there is a gap 625 between the electrically conductive proximal portion 623 of the needle 604 and a distal conductive portion 626 that extends to the distal tip 606. The proximal portion 623 is joined to the distal portion 626 via a ring of electrically insulative material 628 to electrically isolate the proximal portion 623 from the distal portion 626. The inner tube 612 in this embodiment is formed of an electrically conductive material and is connected to the power source at a proximal end with the distal end of the inner tube 612 being electrically coupled to a conductive member 627 which electrically couples the distal portion 626 to the tube 612. A part of the distal portion 626 extending distally beyond the ring 628 forms a second electrode 630 of polarity opposite that of the first electrode 622.

The structure and operation of the device 600 is identical to that of the device 500 except that the inner tube 612 extends distally to the plug 608. Thus, to provide fluid communication between the central passage 614 and the annular channel 616 is facilitated via openings 634 formed in a distal end of the inner tube 612.

FIGS. 7A-7F show various alternate configurations of needles for bi-polar ablation devices similar to devices 500 and 600. These devices differ only in the construction of the needles but are otherwise internally and operationally similar. The needle 700 of FIG. 7A includes a proximal portion 702 formed as an electrically conductive tube (e.g., a stainless steel hypotube) with a first reduced diameter conductive tube 704. An electrically insulative sleeve 706 in turn receives a second reduced diameter conductive tube 708 around a distal end of the tube 704. A distal end of the second reduced diameter tube 708 is received within a distal conductive tube 710. Thus, this needle 700 includes electrically separated electrodes at the tube 710 and a distal portion of the proximal portion 702 with an electrically insulative layer (not shown) defining a proximal end of the electrode formed by the proximal portion 702. A tissue piercing distal tip 712 is formed at a distal end of the distal tube 710.

The needle 720 of FIG. 7B includes a proximal, conductive tube 722 including a reduced diameter distal end 724 (e.g., a swaged hypotube) connected to a distal conductive tube 726 that includes a reduced diameter proximal end 728 (e.g., a swaged hypotube) by an electrically insulative sleeve 730. The tube 726 includes a tissue piercing tip 732 at its distal end and the tubes 722, 726 form the two poles of a bi-polar ablation needle.

As shown in FIG. 7C, the needle 740 includes a proximal electrically insulative tube 742 that receives a first electrode 744 therein. The first electrode 744 includes reduced diameter extensions 746 with a proximal one of the extensions 746 received in the distal end of the tube 742 and a distal extension 746 received within an electrically insulative connector 748. The connector 748 receives a reduced diameter extension 754 of a second electrode 750 therein with the second electrode 750 extending to a tissue piercing tip 752. The first and second electrodes 744, 750, respectively, are connected to opposite poles of a power source to serve as electrodes of a bi-polar ablation device as would be understood by those skilled in the art. The extensions 746 of the first electrode 744 are machined to have a reduced outer diameter compared to a central portion of the first electrode 744. Similarly, the extension 754 of the second electrode 750 is machined to a reduced outer diameter permitting it to be inserted into the distal opening of the connector 748. This permits the needle to have a substantially continuous outer profile while separating the first and second electrodes 744, 750 from one another electrically as desired.

As shown in FIG. 7D, the needle 760 includes a proximal electrically insulative tube 762 that receives a first electrode 764 therein. The first electrode 764 includes reduced diameter extensions 766 with a proximal one of the extensions 766 received in the distal end of the tube 762 and a distal extension 766 received within an electrically insulative connector 768. The connector 768 receives a reduced diameter extension 774 of a second electrode 770 therein with the second electrode 770 extending to a tissue piercing tip 772. The first and second electrodes 764, 770, respectively, are connected to opposite poles of a power source to serve as electrodes of a bi-polar ablation device as would be understood by those skilled in the art. The extensions 766 of the first electrode 764 are swaged to have a reduced outer diameter compared to a central portion of the first electrode 764. Similarly, the extension 774 of the second electrode 770 is swaged to a reduced outer diameter permitting it to be inserted into the distal opening of the connector 768. This permits the needle to have a substantially continuous outer profile while separating the first and second electrodes 764, 770 from one another electrically as desired.

As shown in FIG. 7E, a needle 780 is formed as an electrically insulated member 782 extending from a proximal end (not shown) to a tissue penetrating distal tip 784. Those skilled in the art will understand that this electrically insulated member may be formed of or coated in an electrically insulative material such as the materials described above. The needle 780 includes two conductive sleeves 786 that serve as electrodes for a bi-polar ablation device. Those skilled in the art will understand that the electrodes may be coupled to separate poles of a power source using any known conductors including the inner material of the needle 780 itself when that needle is formed of a conductive material.

As shown in FIG. 7F, a needle 790 is formed as a tube 792 of electrically insulative material with a proximal conductor 794 formed within a first part of the tube 792 and a distal conductor 796 formed in a part of the tube 792 separated circumferentially from the proximal conductor 794. The proximal conductor is electrically coupled to each of a plurality of ring electrodes 798 grouped in a first part of the needle 792 and separated along a length of the needle 792 from a group of distal conductors 800 by a distance selected to isolate the distal conductors electrically from the proximal conductors 798. Each of the distal conductors is electrically connected to the distal conductor 796 which passes by the proximal conductors 798 while electrically insulated therefrom. Those skilled in the art will understand that the proximal electrodes 798 may be coupled to a first pole of a power source while the distal conductors 800 are connected to an opposite pole to operate as electrodes of a bi-polar ablation system.

Variations may be made in the structure and methodology of the present disclosure, without departing from the spirit and the scope of the disclosure. For example, those skilled in the art will understand that any combination of chambers, seals and plungers as described in regard to the devices 100, 200, 300 and 400 may be applied in any of the bi-polar devices described to achieve the same results for tissue collection in a bi-polar device as described in regard to these mono-polar devices and that any of the cooling fluid handling internal structures of any of the devices 100, 200, 300, 400, 500 and 600 may be applied within any of the devices of FIGS. 7A-7F. In another example, any of the devices 100, 200, 300, 400, 500, 600 and 700 may be formed as rigid percutaneous probes as well as the flexible sheathed devices for passage through scopes. In further example, any of the devices of the devices 100, 300, 400, 500, 600 and 700 may also use gas as a cooling option rather than liquid cooling. In another example, the devices 500, 600 may have the option of including the mono-polar biopsy capabilities of the devices 200, 300 and 400. Thus, it is intended that the present disclosure cover the modifications and variations of this disclosure that may be contemplated by a person of skill in the art. For example, any or all of the devices herein may be adapted for percutaneous applications by stiffening the delivery element or through any other modifications as would be apparent to those skilled in the art.

1-14. (canceled)

15. An ablation device, comprising:
a needle extending from a proximal end which, during use, remains outside a body accessible to a user to a tissue penetrating distal tip, the needle defining a lumen extending therethrough to a distal end of the needle;
a first ablation electrode mounted on a distal portion of the needle;
an inner tube received within the lumen of the needle separating the lumen of the needle into an inner cooling fluid supply lumen and an annular fluid return lumen; and
a source of cooling fluid supplying cooling fluid to the cooling fluid supply lumen and withdrawing fluid from the fluid return lumen to remove heat from the first electrode to maintain a temperature of the first electrode below a predetermined threshold temperature.

16. An The ablation device of claim 15, wherein the needle includes a distal opening and a plug sealing the distal opening to direct fluid from the fluid supply lumen into the fluid return lumen.

17. The ablation device of claim 16, wherein the inner tube has a distal end separated proximally from the plug by a distance selected to provide fluid communication between the fluid supply lumen and the fluid return lumen adjacent to the first electrode.

18. The ablation device of claim 16, wherein the inner tube is coupled to the plug and wherein the inner tube comprises at least one opening adjacent the first electrode providing fluid communication with the return lumen.

19. The ablation device of to claim 15, further comprising:
an electrically insulative delivery element extending circumferentially around the needle and receiving the needle therein for movement between a retracted position in which the distal tip of the needle is received within the delivery element and an extended position in which the distal tip of the needle is projected distally beyond a distal end of the delivery element;
wherein the needle is formed of an electrically conductive material, the needle further comprising an electrically insulative coating extending circumferentially therearound, the coating extending along a portion of a length of the needle and having a distal end separated from the distal tip of the needle by a length selected to permit the portion of the needle extending distally from the coating to function as the first electrode, a proximal end of the coating being positioned so that, when the distal tip is projected distally beyond the distal end of the delivery element by a maximum distance, a part of the coating extends into the delivery element.

20. The ablation device of claim 18, wherein the inner tube is slidably received within the needle so that, when the inner tube is withdrawn proximally from the needle, the plug is withdrawn from the distal tip of the needle opening the lumen of the needle to an exterior of the needle so that a tissue sample may be captured therein.

21. The ablation device of claim 20, wherein the plug includes a sealing member extending circumferentially therearound to enhance a seal between the plug and the distal tip of the needle.

22. The ablation device of claim 15, further comprising a wall extending across and sealing a proximal part of the lumen of the needle from a distal portion thereof, the wall being spaced from a distal end of the lumen of the needle by a distance selected to form a tissue sample receiving space in the distal tip of the needle.

23. The ablation device of claim 22, wherein the wall includes an opening therethrough and wherein the device includes a plunger member on a distal side of the wall and a control member extending from the plunger, through the opening to a proximal end accessible to a user so that, tension applied to the control member draws the plunger into contact with the wall to seal the opening and compression applied to the plunger forces the plunger distally away from the wall to drive tissue out of the tissue sample receiving space.

24. The ablation device of claim 15, further comprising a second electrode formed on a distal portion of the needle and separated longitudinally and electrically isolated from the first electrode, the first and second electrodes being coupled to opposite poles of a power source to function as a bi-polar ablation system.

25. The ablation device of claim 24, wherein the needle is formed of an electrically conductive material and includes an electrically insulative coating circumferentially surrounding this electrically insulative coating along at least a portion thereof, and wherein the second electrode is mounted over the electrically insulative coating and the first electrode is mounted on the conductive material of the needle.

26. The ablation device of claim 24, wherein a proximal portion of the needle is formed as a first electrically conductive tube and a distal end of the needle is formed as a second electrically conductive tube, the first and second electrically conductive tubes being joined to one another and electrically isolated from one another by an electrically insulative member.

27. The ablation device of claim 26, further comprising:
an electrically insulative delivery element extending circumferentially around the needle and receiving the needle therein for movement between a retracted position in which the distal tip of the needle is received within the delivery element and an extended position in which the distal tip of the needle is projected distally beyond a distal end of the delivery element; and
an electrically insulative coating extending circumferentially around the first electrically conductive tube from a coating proximal end to a coating distal end, the coating proximal end being positioned so that, when the distal tip is projected distally beyond the distal end of the delivery element by a maximum distance, a part of the coating extends into the delivery element, a part of the first tube extending distally beyond a distal end of the coating forming the second electrode and the second tube forming the first electrode.

28. The ablation device of claim 15, wherein the device is sufficiently flexible to be passed through a natural body lumen until a distal end of the needle reaches a target site within the body.
